(19) United States
(12) Patent Application Publication
(10) Pub. No.: US 2006/0235513 A1
(43) Pub. Date: Oct. 19, 2006

(54) Corneal implants and methods of use

(76) Inventor: Francis W. Price JR., Indianapolis, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/108,505

(22) Filed: Apr. 18, 2005

(51) Int. Cl. A61F 2/14

(52) U.S. Cl. 623/5.12; 623/5.14

(57) ABSTRACT

An implant (20) for reshaping the cornea includes a peripheral ring structure (22) surrounding an inlay member (24) where the inlay member is anchored to the ring structure via a plurality of filaments (26). The implant is surgically positioned with the inlay member generally directly over an ectatic region of the cornea to provide direct re-contouring of the cornea. The inlay member can be provided with a predetermined optical curvature and/or prescriptions to further correct the patient's vision.

### TECHNICAL FIELD

The present invention is generally related to corneal implants and methods of use, and more particularly, but not exclusively, is related to implants for reshaping corneas that have become weaken, thinned or ecstatic, for example due to conditions known as keratoconus and pellucid marginal degeneration, or due to secondary weakening and ectasia after laser refractive surgery or other eye surgery.

### BACKGROUND

Keratoconus (KCN) is a condition where the cornea, the external surface or window of the eye, becomes weakened, thinned, and ectatic. This ectasia causes the cornea to bulge and develop an irregular surface which distorts the optical qualities of the cornea. As the optical quality of the cornea decreases, the quality of vision decreases as well. Current soft contact lenses generally do not significantly improve vision in cases of advanced KCN because the soft contact lenses just confirm to the distortion of the cornea. Because it is not feasible to grind glasses to match the irregular surface of the cornea, most individuals with KCN must wear rigid contact lenses which serve to bridge over the area of the irregularity and provide a new smooth outer surface for the eye. However, hard contact lenses can be uncomfortable and inconvenient to use and maintain.

One surgical treatment option for KCN is a corneal transplant. In this procedure, the patient's diseased corneal tissue is removed and replaced with donor cornea tissue which is then sutured into place. In addition to requiring suitable donor tissue, the transplant surgery involves risks and the recovery period can be long, for example up to one year. A surgical treatment option that is less invasive and does not rely on the availability of donor tissue involves implanting a pair of plastic ring segments in the corneal tissue around the area of irregularity, for example the segments marketed under the brand name INTACS® by Addition Technology, Inc., Fremont, Calif.. Once implanted, these ring segments provide a measure of reinforcement and remodeling of the patient's cornea. However, these implanted ring segments do not directly re-contour the affected area, and as a result the effectiveness of the procedure is limited.

Corneal inlays have been used to change the cornea's ability to focus light. These corneal implants are typically configured like a small contact lens and are implanted in the central cornea at a desired depth. The typically manner of affecting a focusing change is by physically adding shape and contour to the cornea so that the front surface of the cornea changes shape or by having a higher index of refraction than the corneal tissue so that the focusing qualities of the cornea are changed. However, the inherent flexibility of many conventional inlays and/or limitations on their permissible size limit their effectiveness in treating KCN. For example, relatively rigid inlays are typically impermeable to nutrients vital to the survival of the cornea. Thus, while a large diameter rigid inlay might be structurally sufficient to correct the ectasia, the disruption to the natural diffusion processes of the cornea over such a large area could lead to significant necrosis of the corneal tissue.

Accordingly, there is a need for improvements in this area of technology. More specifically, but not exclusively, there is a need for improved implants and surgical techniques that do not depend on the supply of donor corneas and can more directly re-contour specific areas of the cornea. The present invention addresses these and other needs.

### BRIEF DESCRIPTION OF THE FIGURES

Although the characteristic features of this invention will be particularly pointed out in the claims, the invention itself, and the manner in which it may be made and used, may be better understood by referring to the following description taken in connection with the accompanying figures forming a part thereof.

FIG. 1 is a front view of an implant according to one embodiment.

FIG. 2 is a side sectional view of the FIG. 1 device implanted in a cornea.

FIG. 3 is a front view of an implant according to another embodiment.

FIG. 4 is a front view of an implant according to another embodiment.

FIG. 5 is a front view of an implant according to another embodiment.

FIG. 6 is a front view of an implant according to another embodiment.

FIG. 7 is a front view of the FIG. 6 device in a collapsed configuration.

FIG. 8 is a side view of a cornea bulging due to keratoconus.

FIG. 9 is a side view of the FIG. 8 cornea corrected with an implant according to the present invention.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended. Alterations and further modifications in the illustrated devices, and such further applications of the principles of the invention as illustrated herein are contemplated as would normally occur to one skilled in the art to which the invention relates.

In one form, the present invention provides an implant useful for reshaping ectatic regions of the cornea. The implant utilizes a peripheral ring (or a number of arcurate segments) and an interior member (e.g. an inlay) that is anchored to the ring via a series of thin connecting members (e.g. filaments). To re-contour the cornea, the implant is positioned with the inlay generally over the ectasia and the ring generally surrounding the ectasia. It is expected that, as compared to use of a ring alone, the relatively direct re-contouring provided by the inlay enables greater control over the final curvature or shape of the cornea. Furthermore, the structure imparted to the inlay by the peripheral ring (or arcurate segments) permits the inlay to be of smaller size and/or constructed from a more flexible material than would be possible if inlay were to be used alone.

It is to be understood that, as used herein, a ring refers to a body defining an open center, whether constructed of linear segments, segments of constant radius, or segments where the radius of curvature varies. Accordingly, rings can be circular, oval, oblong or asymmetrical and their size is given by a characteristic dimension measured along a line passing through the center of the circle, oval etc. For example the characteristic dimension of a circle is its diameter. A portion of a ring includes any arcurate segment whether or not that arcurate segment is a part of a complete ring. It is also to be appreciated that rings adapted to be implanted in the eye will typically have a spherical or aspherical curvature to substantial conform to the curvature of the globe of the eye, such as show in connection with the scleral expansion bands described in application Ser. No. 10/462,366, Publication No. 2004/0034415.

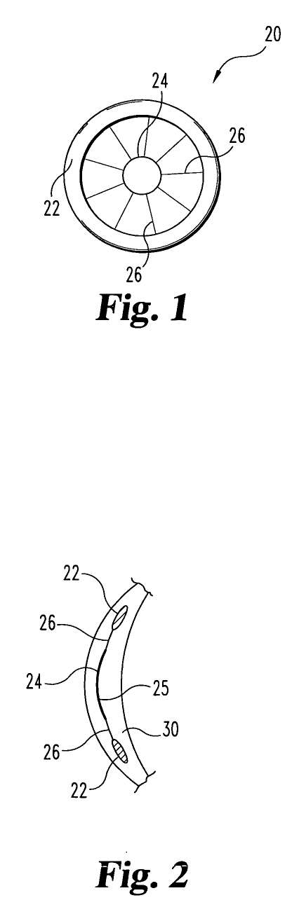

Turning now to FIG. 1 an implant 20 according to one embodiment is depicted. The implant 20 includes a peripheral ring 22 surrounding an interior disc shaped member 24. In this first embodiment, a plurality of filaments 26 span radially between the member 24 and the ring 22 in a spoke like pattern. The ends of the filaments 26 are connected to the ring 22 and the member 24 respectively in any suitable fashion, for example by tying, sutures or glue. In use, the implant 20 is surgically implanted into a cornea 30, as illustrated in the cross section of FIG. 2, with the inside surface 25 of the member 24 cradling the affected area of the cornea 30.

The ring 22 maintains the filaments 26 in tension so as to support the member 24 as it directly reshapes an ectatic area of the cornea 30. The ring 22 can also assist in remodeling the cornea by the indirect method of INTACTS®, e.g. by flattening the surrounding corneal tissue. However, the ring 22 need not function to flatten the surrounding corneal tissue, and the size and configuration of the ring can generally be selected to control the amount of flattening attributable to the ring 22. For example, INTACTS® currently in use are in the 6 to 8 mm size range, for example having an inner diameter of 6.77 mm and outer diameter of 8.1 mm with a hexagonal cross-section. Because the present implant 20 does not rely solely on the presence of the ring 22 to flatten the surrounding corneal tissue, there is a significant flexibility in choice of size and configurations. For example, while the ring 22 can be of any suitable size and configuration, it is expected that certain embodiments will employ a ring in the 8-12 mm size range, for example having a characteristic inner dimension of approximately 9 mm and a characteristic outer dimension of 10 mm (e.g. for a circle, the inner and outer diameters).

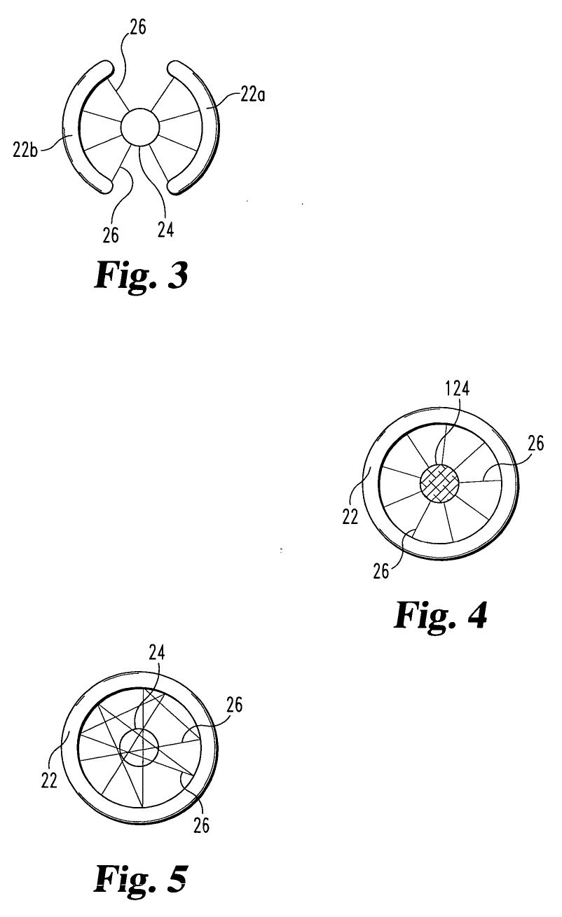

One or more ring segments can be used in place of ring 22. For example, FIG. 3 depicts an implant having arcurate segments 22a and 22b which only partially encircle or surround the member 24. Applications with three or four or more arcurate segments are also contemplated.

In many applications, the function of the member 24 will be primarily to provide support to the affected area of the cornea, and the member 24 can be constructed of any biocompatible material having suitable mechanical properties for that purpose. For example FIGS. 1, 2, and 3 illustrate a member 24 that is formed of a solid material as is commonly done with conventional corneal inlays. FIG. 4 illustrates an alternative construction where the interior member 124 is formed from a mesh.

The member 24 can be rigid or flexible. In one form, the member 24 is as flexible as a soft contact lens and substantially relies on the ring 22 to maintain its shape and to reshape the cornea. In another form, the member 24 has rigidity comparable to a hard contact lens and can maintain its shape even without being connected to the outer ring 22.

In other applications, the member 24 is constructed to achieve additional objectives, such as to improve the focusing quality of the eye to correct nearsightedness, farsightedness, astigmatism or presbyopia. In these applications, an inlay having a predetermined optical prescription and/or having a predetermined optical curvature (e.g. to achieve a specified diopter correction) may be employed. In other applications the inlay can be plano refractive.

The size and shape of the member 24, 124 can vary based on the particular application. Typical members 24, 124 will be disc shaped (as seen viewed from the perspective of FIG. 1) and in the range of 4-6 mm in diameter. Because the cornea is avascular and relies on diffusion for transport of oxygen and nutrients, the interior member should be constructed so as not to unduly interfere with the diffusion of oxygen and nutrients which can lead to necrosis of the cornea. Use of a mesh or weave pattern (such as member 124) is one mechanism for accomplishing this. Another way to avoid causing necrosis is to have the member 24 be of relatively small size (diameter) such that any restriction of the diffusion of oxygen or nutrients directly through the member is adequately offset by diffusion through the surrounding tissue. Where the member 24 is provided by a relatively large diameter inlay, a suitable technique is to construct the inlay from a material that is sufficiently permeable to oxygen and glucose to avoid causing necrosis of the cornea. Suitably permeable materials for large inlays include high water content materials such as used in soft contact lenses, hydrogels and collagen-polymers mixtures (termed colamers).

The filaments 26 function to transfer the structural rigidity of the ring 22 to the interior member 24. In other words, they anchor the interior member 24 to the outer ring 22, which in many cases would be stably fixated, to help counteract the ectasia of the cornea. The filament 26 or connecting member can be constructed of any thin biologically compatible material with suitable resistance to stretching including wires and rods. While the purpose of the filaments 26 is to produce a radial component of force to stabilize the interior member, they need not be in a spoke pattern. For example, FIG. 5 depicts an implant where the filaments are in an interlocking web pattern. The FIG. 5 implant also illustrates the filaments overlaying the interior member 24, as opposed to being connected about its periphery, and this implementation could be applied where the interior member is a mesh.

Implants according to the present invention can be surgically implanted as would occur to those of skill in the art. One suitable technique is to perform a lamellar (layered) dissection of the cornea separating the anterior from the posterior layers. The depth of the dissection in either percent of corneal thickness or absolute thickness can vary. In one procedure, upon insertion, the ring 22 or one or more of the ring segments 22a and 22b are sutured in place. In another, the ring and or ring segments are left unconstrained.

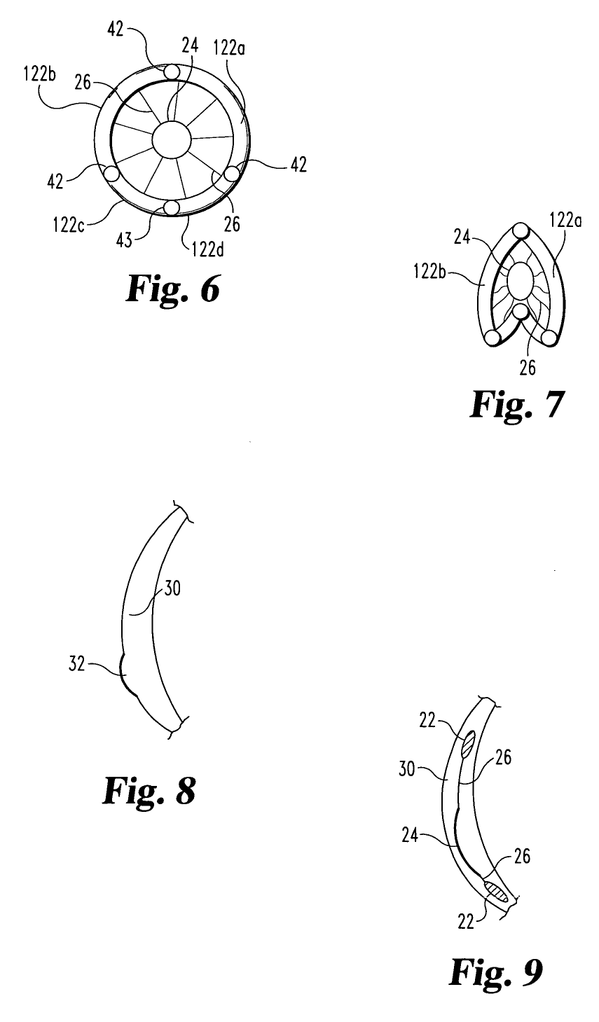

The implant can be implanted as a preassembled whole or implanted in sections and assembled in the cornea. FIGS. 6 and 7 illustrate a variation designed to assume an expanded configuration (FIG. 6) during use and a collapsed configuration (FIG. 7) during implantation. The ability to assume a collapsed configuration during implantation reduces the size of the incision needed. The ring in the FIG. 6 embodiment is constructed from four segments, two larger 122a, 122b and two smaller 122c, 122d. The ring segments are connected by hinges 42 and 43. Hinges 43 is a locking hinge that, when unlocked, collapsed towards the interior of the ring to allow the device to assume the collapsed configuration of FIG. 7.

The peripheral ring 22 or ring segments 22a, 22b are made of a material that is sufficiently rigid to exert a force on the adjacent tissue and/or to apply tension to the thin connecting members sufficient to achieve deformation of the corneal tissue as described herein. Such materials are well-known in the surgical art and include suitable metals, ceramics, and plastics. Preferably, the ring or ring segments are constructed of a thin transparent material, such as employed in contact lenses and the like. Suitable materials include physiologically inert materials such as polymethylmethacrylate (PMMA), polyethylene, polypropylene, poly(tetrafluoroethylene), polycarbonate, silicone resins, and combination materials with collagen, and the like. The ring and ring segments may be manufactured by any conventional technique appropriate to the material used, such as machining, injection molding, heat molding, compression molding and the like.

Each of the various components of the implant may optionally be used to deliver chemicals, medications, vitamins or other therapeutic compositions to the eye. This may be accomplished by having the respective therapeutic composition embedded in or coated on the respective component. Examples of such materials that can be coated or embedded in the implant include Riboflabin (vitamine B2), corticosteroids, growth factors, anti-neovascular signaling factors, non-steroidal anti-inflammatory drugs, collagen cross linking chemicals, or anti-metabolite drugs such as Mitomycin-C or any other drug suitable for decreasing scar formation, neovascularization, inflammation, or for enhancing the structural integrity of the cornea.

Alterations of the color, surface coating, surface finish and/or composition of the various components can also be employed to reduce any undesirable side effects on the patent's vision, such as glare, reflections or light scatter from the components of the implant.

It is to be appreciated that, while in some cases the interior member will be in the center of the ring or ring segments, it can be positioned wherever necessary. For example, the pupil and center of vision through the cornea is often times not located directly in line with the center of the cornea, and in these cases, the interior member 24 may be offset to lie over the center of the pupil or center of the visual axis in the eye. In another example, FIG. 8 illustrates a cornea having a bulge that is not in the central cornea, but inferior to it. Bulges near the lower edge of the cornea or in the peripheral or mid-peripheral cornea are often found in cases of pellucid marginal degeneration, a condition related to KCN. The implant constructed to correct this condition, shown implanted in the cross section of FIG. 9, has the member 24 substantially offset from the center of the ring 22. In other words, the member 24 is substantially closer to one portion of the ring 22 than it is to another portion.

### CLOSURE

While the invention has been illustrated and described in detail in the drawings and foregoing description, the same is to be considered as illustrative and not restrictive in character. Only certain embodiments have been shown and described, and all changes, equivalents, and modifications that come within the spirit of the invention described herein are desired to be protected. Any experiments, experimental examples, or experimental results provided herein are intended to be illustrative of the present invention and should not be considered limiting or restrictive with regard to the invention scope. Further, any theory, mechanism of operation, proof, or finding stated herein is meant to further enhance understanding of the present invention and is not intended to limit the present invention in any way to such theory, mechanism of operation, proof, or finding. Thus, the specifics of this description and the attached drawings should not be interpreted to limit the scope of this invention to the specifics thereof. Rather, the scope of this invention should be evaluated with reference to the claims appended hereto. In reading the claims it is intended that when words such as “a”, “an”, “at least one”, and “at least a portion” are used there is no intention to limit the claims to only one item unless specifically stated to the contrary in the claims. Further, when the language “at least a portion” and/or “a portion” is used, the claims may include a portion and/or the entire items unless specifically stated to the contrary. Finally, all publications, patents, and patent applications cited in this specification are herein incorporated by reference to the extent not inconsistent with the present disclosure as if each were specifically and individually indicated to be incorporated by reference and set forth in its entirety herein.

What is claimed is:

1. A corneal implant comprising:
an interior member adapted to be implanted in a cornea and to reshape to an area of the cornea;
at least one curved body spaced from the interior member and at least partially surrounding the interior member; and
a plurality of connecting members spanning between the curved body and the interior member;
wherein the connecting members impart structural support to the interior member to remodel the cornea with the interior member when the device is implanted.

2. The implant of claim 1 wherein the at least one curved body has a characteristic size of between about 8 mm and about 12 mm.

3. The implant of claim 1 wherein the interior member is an inlay.

4. The implant of claim 3 wherein the inlay is permeable to oxygen and glucose when implanted in the cornea.

5. The implant of claim 4 wherein the at least one curved body is substantially more rigid than the inlay.

6. The implant of claim 1 wherein the interior member is an interconnected mesh.

7. The implant of claim 1 wherein the plurality of connecting members overlap each other.

8. The implant of claim 7 wherein the plurality of connecting members form an interconnecting mesh.

9. The implant of claim 8 wherein the interior member comprises an inlay and the interconnecting mesh covers the inlay.

10. The implant of claim 1 wherein the plurality of connecting members extend radially from the interior member to the at least one curved body.

11. The implant of claim 1 wherein the at least one curved body forms a ring completely surrounding the interior member.

12. The implant of claim 11 wherein the ring is composed of a plurality of interconnected segments.

13. The implant of claim 12 wherein the interconnections are unequally spaced about the ring.

14. The implant of claim 12 wherein the segments are positionable into a compact configuration during implantation.

15. The implant of claim 1 wherein the plurality of connecting members are under tension.

16. The implant of claim 3 wherein the inlay defines a prescription.

17. The implant of claim 3 wherein the inlay is plano refractive.

18. The implant of claim 3 wherein the inlay has a preselected curvature.

19. The implant of claim 3 wherein the at least one curved body defines a center and the interior member is offset from the center defined by the at least one curved body.

20. A method comprising:
reshaping the cornea of a patient by implanting in the cornea at least one curved body at least partially surrounding an interior member wherein a plurality of tensioned members span between the interior member and the at least one curved body.

21. The method of claim 20 wherein the interior member defines a preselected optical prescription.

22. The method of claim 20 wherein the interior member defines a preselected optical curvature.

23. The method of claim 20 wherein the patient has been diagnosed with at least one of keratoconus and pellucid marginal degeneration.

24. The method of claim 20 wherein the implanting is via a lamellar dissection of the cornea.

25. A corneal implant comprising:
an interior member adapted to receive an ecstatic portion of a cornea when implanted in an eye;
a plurality of thin members contacting the interior member; and
at least one curved member forming a substantial portion of a perimeter around the interior member and adapted to apply tension to the thin members to at least partially correct the ectasia with the interior member when implanted in the eye.

26. The implant of claim 25 wherein the interior member is permeable to oxygen.

27. The implant of claim 26 wherein the interior member is permeable to glucose.

28. The implant of claim 25 wherein the interior member is an inlay.

29. The implant of claim 28 wherein the inlay has at least one of a preselected optical curvature and a preselected optical prescription.

30. A corneal implant comprising:
a peripheral body forming at least a portion of a ring;
an interior member in the interior of the ring or portion thereof; and
a plurality of tensioned members spanning between the peripheral body and the interior member, the tensioned members having a thickness substantially less than a thickness of the peripheral body.

31. The implant of claim 30 wherein the ring or portion thereof has a characteristic outer dimension less than about 12 mm.

32. The implant of claim 30 wherein the ring or portion thereof has a characteristic outer dimension greater than about 8 mm

33. The implant of claim 32 wherein the ring or portion thereof has a characteristic outer dimension less than about 12 mm.

34. The implant of claim 30 wherein the ring or portion thereof is substantially circular.

35. The implant of claim 30 wherein the ring or portion thereof is not substantially circular.

36. The implant of claim 30 wherein at least one of peripheral body, the interior member or the tensioned members is coated or embedded with at least one therapeutic composition.

37. The implant of claim 36 wherein the therapeutic composition is selected from corticosteroids, growth factors, anti-neovascular signaling factors, non-steroidal anti-inflammatory drugs, collagen cross linking chemicals, and anti-metabolite drugs.
